(19) United States
(12) Patent Application Publication
(10) Pub. No.: US 2026/0265651 A1
(43) Pub. Date: Sep. 10, 2026

(54) ORGAN-MIMICKING ORGAN-ON-A-CHIP AND DYNAMIC ORGAN MIMICKING SYSTEM FOR DISEASE MODELING

(71) Applicant: POSTECH RESEARCH AND BUSINESS DEVELOPMENT FOUNDATION, Pohang-si (KR)

(72) Inventors: Junmin LEE, Pohang-si (KR); Taehoon LEE, Pohang-si (KR); Geonwoo KIM, Pohang-si (KR); Giheon HA, Pohang-si (KR)

(73) Assignee: POSTECH RESEARCH AND BUSINESS DEVELOPMENT FOUNDATION, Pohang-si (KR)

(21) Appl. No.: 19/555,202

(22) Filed: Mar. 3, 2026

(30) Foreign Application Priority Data
Mar. 4, 2025 (KR) 10-2025-0027749
Oct. 24, 2025 (KR) 10-2025-0156124

(51) Int. Cl. C12M 3/00; C12M 1/00; C12M 1/12; C12M 1/34; C12M 1/42; C12M 3/06

(52) U.S. Cl. CPC C12M 21/08; C12M 23/16; C12M 23/26; C12M 25/02; C12M 35/02; C12M 35/04; C12M 41/34

(57) ABSTRACT

An Organ-Mimicking Organ-on-a-Chip is provided. The Organ-Mimicking Organ-on-a-Chip comprises a cell culture layer and a platform including a membrane part on which the cell culture layer is seated and an air chamber providing deformation force to the membrane part. The cell culture layer is capable of two-dimensional or three-dimensional deformation according to the deformation of the membrane part. The air chamber provides periodic compression and tensile stimulation through injection and discharge of gas, thereby mimicking dynamic mechanical movement occurring in the actual human body. Various embodiments include configurations with lateral deformation, vertical deformation, and combinations thereof. The device can be used for drug evaluation, disease modeling, and tissue engineering applications.

### TECHNICAL FIELD

The present invention relates to Human Organ Mimicking, which is a technology for mimicking the structure and function of human organs in vitro.

The present invention also relates to In Vitro Tissue Environment Models, which is a technology for reproducing the microenvironment of human tissue in a laboratory environment.

The present invention relates to Cell Culture in Physiologically Relevant Environments, which is a technology for culturing cells in conditions similar to the actual in vivo environment.

The present invention also relates to Drug Evaluation, which is a platform technology for testing the efficacy and toxicity of new drugs.

The present invention relates to Microfluidics, which is the development of biomimetic systems using microfluidic technology.

The present invention also relates to Bioengineering, which is a field for engineering the design and fabrication of biological systems.

Finally, the present invention relates to Regenerative Medicine, which is a research field for restoring or replacing the functions of damaged tissues or organs.

### BACKGROUND

The present invention relates to the fields of Human Organ Mimicking, In Vitro Tissue Environment Models, cell culture in physiologically relevant environments, and Drug Evaluation. Recently, in this technical field, organoid and organ-on-a-chip technologies that mimic the complex structure and function of human organs are rapidly developing, and in particular, attention is being paid to mimicking technologies for various organs in which repetitive physical stimulation plays an important role.

For example, mimicking technologies for organs with periodic contraction and expansion such as lungs, organs with beating such as hearts, or organs with peristaltic movement such as intestines are being actively researched. The development of biomimetic systems using advanced technologies such as 3D printing and Microfluidics is in progress, and through this, efforts are being made to reproduce the unique dynamic environment of each organ. Also, interest in developing personalized organ cell models for patient-specific medical research is increasing.

However, in the conventional technology, there was an absence of technology that can accurately control complex physical stimulation given to various organ cells while simultaneously obtaining a large amount of experimental results, and due to this, there were limitations in the efficiency and reproducibility of experiments. Also, the chemical composition of the cell culture layer was uniform, so the range of change in physical stimulation was limited, and this gave difficulty in accurately mimicking the various physical properties of actual human tissue.

Most in vitro organ models cultured cells in a static environment and could not reproduce the dynamic mechanical stimulation occurring in actual organs, and the existing 2D cell culture model could not reflect the 3D structure of organs, showing limitations in predicting drug responses. In the case of animal models, there were limitations in accurately predicting the drug effect or toxicity on human organs due to species differences in organ structure and function.

Due to these technical trends and limitations of conventional technology, the development of various organ mimicking systems that are more sophisticated and physiologically relevant was required, and in particular, the necessity of a new organ-on-a-chip platform capable of high-efficiency experiments while precisely controlling various physical stimulations suitable for the characteristics of each organ emerged.

One prior art reference is Republic of Korea Registered Patent No. 10-1569619, registered on Nov. 10, 2015.

### SUMMARY

A first aspect of the present invention provides an Organ-Mimicking Organ-on-a-Chip capable of mimicking physical movement characteristics similar to a living body.

A second aspect of the present invention provides a method for manufacturing an Organ-Mimicking Organ-on-a-Chip capable of implementing physical properties similar to living tissue.

A third aspect of the present invention provides a Dynamic Organ Mimicking System capable of precisely controlling dynamic mechanical stimulation of human organs and capable of constructing a personalized organ model using patient-specific cells.

A fourth aspect of the present invention provides a drug administration device for Dynamic Organ Mimicking System.

According to one embodiment, an Organ-Mimicking Organ-on-a-Chip comprises: a cell culture layer; and a platform including a membrane part on which the cell culture layer is seated and an air chamber providing deformation force to the membrane part.

In one embodiment, the cell culture layer is capable of two-dimensional or three-dimensional deformation according to the deformation of the membrane part.

In one embodiment, the platform is manufactured by mixing polydimethylsiloxane (PDMS) and a curing agent.

In one embodiment, the membrane part is more flexible than the air chamber.

In one embodiment, the membrane part is manufactured by mixing polydimethylsiloxane (PDMS) and a curing agent in a composition ratio of 25:1 to 35:1.

In one embodiment, the air chamber is manufactured by mixing polydimethylsiloxane (PDMS) and PDMS curing agent in a composition ratio of 8:1 to 12:1.

In one embodiment, the air chamber includes a first air chamber and a second air chamber, wherein the first air chamber and the second air chamber are located on both sides of the membrane part to provide deformation force in a lateral direction to the cell culture layer.

In one embodiment, the Organ-Mimicking Organ-on-a-Chip further comprises a third space and a fourth space on both sides of the membrane part, wherein the third space and the fourth space induce more uniform deformation in the cell culture layer.

In one embodiment, the air chamber is located at a lower end of the cell culture layer, and a media chamber containing liquid medium in which material exchange between the membrane part and cell layer occurs is provided on the air chamber so that the cell culture layer can be deformed in a vertical direction.

In one embodiment, the Organ-Mimicking Organ-on-a-Chip includes a plurality of the air chambers, the membrane parts, and the media chambers.

In one embodiment, sizes of the air chambers are different from each other, and the plurality of membrane parts vary according to the size of the air chamber.

In one embodiment, each of the air chambers is independently controlled to simultaneously provide different physical stimulation to the plurality of cell culture layers.

In one embodiment, the Organ-Mimicking Organ-on-a-Chip further comprises a microfluidic supply unit that supplies culture solution containing nutrients and oxygen necessary for the cell culture layer.

In one embodiment, the Organ-Mimicking Organ-on-a-Chip further comprises a gas exchange unit for controlling oxygen (O₂) and carbon dioxide (CO₂) concentration of the cell culture layer.

In one embodiment, the Organ-Mimicking Organ-on-a-Chip further comprises an electrical/mechanical stimulation unit that provides electrical or mechanical stimulation for growth and functional improvement of the cell culture layer.

In one embodiment, the air chamber, a microfluidic system, a gas exchange system, and the electrical/mechanical stimulation unit are configured to mimic disease-specific breathing patterns, and the air chamber reproduces physiological conditions of a specific disease by controlling respiratory cycle and pressure change.

The Organ-Mimicking Organ-on-a-Chip according to the present invention has the effect of more accurately mimicking the structure of actual human tissue by providing periodic compression and tensile stimulation through an air chamber so that the cell culture layer can be deformed in 2D or 3D.

The Organ-Mimicking Organ-on-a-Chip and manufacturing method according to the present invention can implement physical properties similar to actual human tissue by using hydrogel with strong ductility as the material of the membrane and can apply a wide range of physical stimulation.

By arranging a plurality of experimental conditions in parallel within a single chip, experimental results of multiple conditions can be obtained simultaneously.

The Organ Mimicking System according to the present invention provides physical stimulation that can be precisely controlled through an air injection/discharge system.

The drug administration device for Organ Mimicking System according to the present invention can experiment with the effect by wirelessly controlling the administration timing of various types of drugs to the cell culture layer of the organ mimicking system.

### BRIEF DESCRIPTION OF THE DRAWINGS

FIG. 1 is a perspective view showing an Organ-Mimicking Organ-on-a-Chip according to a first embodiment including a platform having a cell culture layer, a membrane part, and an air chamber providing deformation force.

FIG. 2A is a side view of FIG. 1.

FIG. 2b is a photograph of FIG. 1.

FIG. 3 is an operational view showing an operational state.

FIGS. 4A-4C show a second embodiment of an Organ-Mimicking Organ-on-a-Chip when a width of a second membrane part is narrowed in the first embodiment. FIG. 4A is a plan view,

FIG. 4B is a side view, and FIG. 4C is an actual photograph.

FIG. 5 is a cross-sectional view showing an Organ-Mimicking Organ-on-a-Chip according to a third embodiment including a platform having a cell culture layer, a membrane part, and an air chamber providing deformation force.

FIG. 6a is a schematic view of an organ-on-a-chip according to a fourth embodiment of the present invention, and FIG. 6b is a photograph thereof.

FIG. 7 shows culture conditions of a cell culture layer.

FIG. 8 is a visual representation of types of mechanical stimulation that can be applied to a cell culture layer, showing patterns and changes of Stretch and Compression stimulation.

FIG. 9 is a graph evaluating cell death change due to mechanical stimulation applied to cells.

FIG. 10 is a view showing that various contractions and expansions of a cell culture layer can be confirmed as a result of experiments on a plurality of cell culture layers with different membrane part sizes according to a size of an air chamber.

FIG. 11 is a flowchart showing a manufacturing process of an organ-mimicking organ-on-a-chip according to a second aspect of the present invention.

FIGS. 12 and 13 are schematic diagrams of a pressure supply unit capable of periodically applying gas and vacuum.

FIG. 14 illustrates examples of types of mechanical stimulation that can be applied by generating various waveforms by a controller.

FIG. 15 is a schematic diagram of a drug administration device.

FIG. 16 is an exploded perspective view and cross-sectional view showing a three-layer stacked structure of an organ-on-a-chip, a photograph of an actually manufactured device, and dimensions of each layer according to one embodiment of the present invention.

FIG. 17 is a microscope image and graph showing experimental results of an optimal mixing ratio of GelMA and Gelatin according to one embodiment of the present invention.

FIG. 18 is a conceptual diagram showing a principle of real-time mechanical property measurement through imaging and deformation modeling of a PDMS membrane and an alveolar membrane according to one embodiment of the present invention.

FIG. 19 is a graph showing simulation verification results of a mechanical property calculation formula according to one embodiment of the present invention.

FIG. 20 is a reference table for determining a fibrosis progression state according to Young's modulus according to one embodiment of the present invention.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Before describing the present invention in detail, it should be understood that the terminology used in the present specification is for the purpose of describing specific embodiments only and is not intended to limit the scope of the present invention. All technical and scientific terms used in the present specification have the same meaning as commonly understood by one of ordinary skill in the art to which this invention belongs, unless otherwise stated.

Throughout the present specification, unless otherwise stated, the term “comprise,” “comprises,” or “comprising” means including the mentioned element, step, or group of elements and steps, and is not used in the sense of excluding any other element, step, or group of elements or group of steps.

In the drawings, the width, length, thickness, angle, etc. of components may be exaggerated for convenience. When one component is described as being “on,” “above,” “under,” or “below” another component, this includes not only the case where it is directly on, above, under, or below another component, but also the case where there is another component in between.

Various embodiments of the present invention can be combined with any other embodiments unless there is a clear indication to the contrary. Any feature indicated as preferred or advantageous may be combined with any other feature and features indicated as preferred or advantageous.

### First Aspect

An Organ-Mimicking Organ-on-a-Chip according to a first aspect includes: a cell culture layer; and a platform having a membrane part on which the cell culture layer is seated and an air chamber providing deformation force to the membrane part.

The cell culture layer is designed to mimic the structure and function of actual organs. The cell culture layer includes at least one cell layer and may optionally include a media layer.

The composition of the cell culture layer may vary depending on the characteristics of the organ or tissue to be mimicked. The cell culture layer is composed of cells that perform the main functions of the relevant organ. For example, when mimicking lung tissue, lung epithelial cells and lung endothelial cells are used. Epithelial cells are cultured on the surface in contact with air to act as the first barrier for gas exchange, and endothelial cells are cultured on the surface in contact with media to mimic blood vessel walls. These cells perform functions in actual organs and show various physiological reactions in response to physical stimulation.

The media layer mimics the vascular function of actual organs. In the case of a lung-on-chip model, this layer performs roles such as gas exchange, nutrient supply, metabolite removal, and blood flow mimicking. Oxygen is delivered to cells and carbon dioxide is removed through the media layer, and nutrients necessary for cells are supplied. The media layer can also be utilized in drug delivery and response research and plays an important role in evaluating the effect or toxicity of drugs on lung tissue.

When the cell culture layer according to the present invention mimics lung tissue, lung epithelial cells and lung endothelial cells can be used. Epithelial cells can be cultured on the surface in contact with air, and endothelial cells can be cultured on the surface in contact with media.

The platform is a core element that provides the basic structure of the organ-on-a-chip, and is an entire structure including the membrane part and the air chamber. It is manufactured by mixing polydimethylsiloxane (PDMS) and PDMS curing agent in a ratio of 9:1 to 35:1, thereby providing the necessary flexibility.

One of the main features of the platform is that it can reproduce dynamic mechanical stimulation. This is implemented through an air chamber. The air chamber provides periodic compression and tensile stimulation through the injection and discharge of gas, thereby mimicking the dynamic mechanical movement occurring in the actual human body.

The operating principle of the air chamber is to periodically repeat pressure increase due to gas injection into the air chamber and pressure decrease due to discharge to stretch and relax the membrane part.

The air chamber can control minute pressure changes using a pressure sensor and a precision valve, so the force applied to the membrane part can be controlled very precisely. This is important for accurately mimicking complex physiological conditions.

The membrane part is a section that is two-dimensionally or three-dimensionally deformed according to the stretching and compression of the air chamber. For deformation more similar to the human body, the membrane part is manufactured more flexibly than other parts of the platform, that is, the air chamber. For example, it is preferable that the ratio of PDMS and curing agent is set to 30:1 in the seating part and 10:1 in the remaining parts. The thickness of the seating part is set within a range of 100 to 1000 μm, which is for the optimal balance between structural stability and flexibility.

According to the two-dimensional or three-dimensional deformation of the membrane part, the cell culture layer is also capable of two-dimensional or three-dimensional deformation.

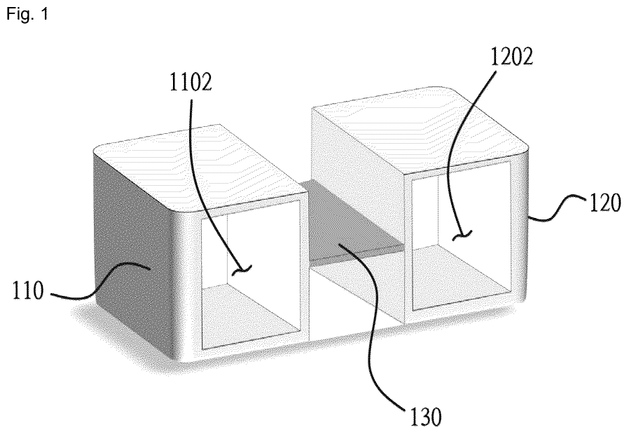

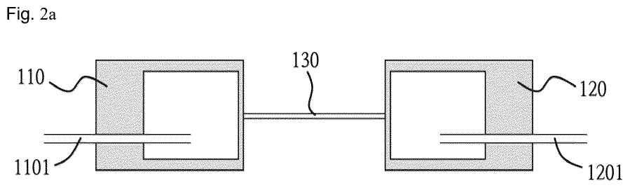

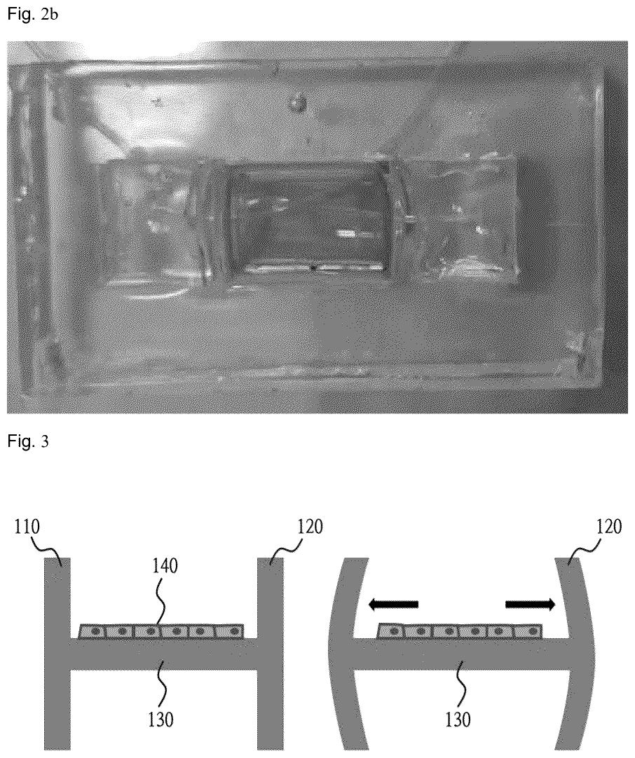

Reference is now made to FIG. 1, which is a perspective view showing an Organ-Mimicking Organ-on-a-Chip according to a first embodiment including a platform having a cell culture layer, a membrane part, and an air chamber providing deformation force. FIG. 2A is a side view of FIG. 1, and FIG. 2B is a photograph of FIG. 1. FIG. 3 is an operational view showing an operational state. According to this embodiment, the first embodiment has a first air chamber 110 having a first space 1102 and a second air chamber 120 having a second space 1202 on the left and right. A membrane part is located between these two chambers, and a first cell culture layer 140 is provided on the first membrane part 130. A microfluidic supply unit 150, a gas exchange unit 160, and an electrical/mechanical stimulation unit 170 are provided on one surface of the first cell culture layer.

The first embodiment is suitable for mimicking movement in which the first cell culture layer 140 is deformed in the lateral direction. The first air chamber 110 and the second air chamber 120 are sealed spaces and are provided with a first tube 1101 and a second tube 1201. Lateral deformation force is provided to the first membrane part 130 through injection and discharge of air through the first tube 1101 and the second tube 1201 to mimic in vivo mechanical stimulation. The first membrane part 130 is made of a flexible material and plays a role of effectively transmitting the force from the first air chamber 110 and the second air chamber 120 to the first cell culture layer 140.

The first cell culture layer 140 includes a cell layer and may include a media layer as needed. It provides an environment for cell attachment, growth, and differentiation and mimics the structure and function of actual tissue or organs. Through this structure, lateral forces are applied to the membrane part by simultaneous or alternating operation of the left and right first air chamber 110 and second air chamber 120. This force is transmitted to the cell culture layer through the membrane part, so that the cells receive mechanical stimulation similar to what they experience in actual living bodies.

The microfluidic supply unit 150 plays a role of supplying culture solution containing nutrients and oxygen necessary for the cell culture layer. The microfluidic supply unit 150 circulates culture solution containing nutrients, oxygen, growth factors, etc. necessary for cell growth and function maintenance at a constant speed, thereby supporting metabolic activity of cells and removing waste products.

In one embodiment, the microfluidic supply unit can supply culture solution to the cell culture layer through microchannels arranged to be adjacent to the cell culture layer to provide an environment similar to actual living tissue to the cell culture layer.

The gas exchange unit 160 plays a role of controlling the concentration of oxygen (O₂) and carbon dioxide (CO₂) around the cell culture layer. It can provide an environment more similar to actual organs by promoting cell metabolism of the cell culture layer through oxygen supply and maintaining pH balance in the culture solution through carbon dioxide discharge.

In one embodiment, the gas exchange unit configures a closed circuit composed of a space disconnected from the external atmosphere for the Organ-Mimicking Organ-on-a-Chip including the cell culture layer, and can control the concentration of oxygen (O₂) and carbon dioxide (CO₂) around the cell culture layer by inspiring or expiring oxygen (O₂) and carbon dioxide (CO₂) through a flow path leading to the inside and outside of the closed circuit.

The electrical/mechanical stimulation unit 170 is a system that provides electrical or mechanical stimulation for cell growth and functional improvement. Specifically, it has the effect of promoting metabolism of cells in the cell culture layer by applying mechanical signals (pressure, tension) or electrical signals to the cell culture layer, and as a result, promoting cell growth.

In one embodiment, the electrical/mechanical stimulation unit 170 is provided between the cell culture layer 130 and the first membrane part and can activate specific cells by controlling electrical signals applied to the cell culture layer. It plays a role of providing an environment similar to the in vivo environment by inducing lung tissue cells to repeatedly stretch and contract through mechanical stimulation.

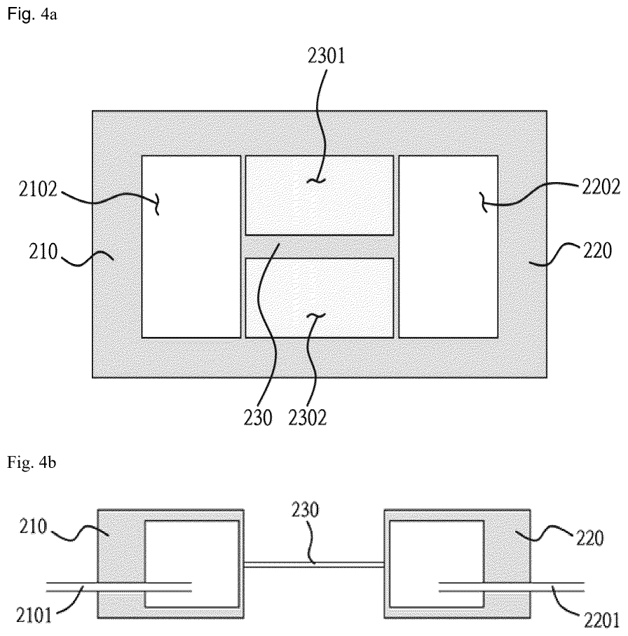

Reference is now made to FIG. 4, which shows a second embodiment of an Organ-Mimicking Organ-on-a-Chip when the width of a second membrane part 230 is narrowed in the first embodiment. FIG. 4A is a plan view, FIG. 4B is a side view, and FIG. 4C is an actual photograph.

When lateral force is transmitted, the area of the second membrane part 230 greatly affects the force transmission pattern. When the area is large, the force is distributed over a wide area, so the intensity of the force per unit area becomes relatively low. The degree of deformation at the center and edge of the second membrane part 230 may be different, so the stimulation received by cells may vary depending on the position.

The overall deformation amount may be large, but the local deformation degree may be relatively small. Due to the large area, the force may be transmitted gradually, so there may be less sudden deformation. On the other hand, when the area is narrow, the force is concentrated in a narrow area, so the intensity of the force per unit area becomes relatively high. Due to the small area, there is a high possibility of more uniform deformation overall.

The overall deformation amount may be small, but the local deformation degree may be relatively large. Due to the narrow area, the force may be transmitted immediately, so sudden deformation may occur. Due to this difference, the intensity and pattern of mechanical stimulation experienced by cells may vary depending on the area of the membrane part, and this may affect cell reactions and behavior.

Therefore, the second embodiment is an embodiment for mimicking a living body in which the force is concentrated in a narrow area compared to the first embodiment, so the intensity of the force per unit area becomes relatively high, and due to the small area, more uniform deformation occurs overall. In this case, additional third space 2101 and fourth space 2102 are further provided on the side of the second membrane part 230 between both air chambers. Thereby, the second membrane part 230 is provided at a narrow interval, so that high force and uniform stimulation can be applied.

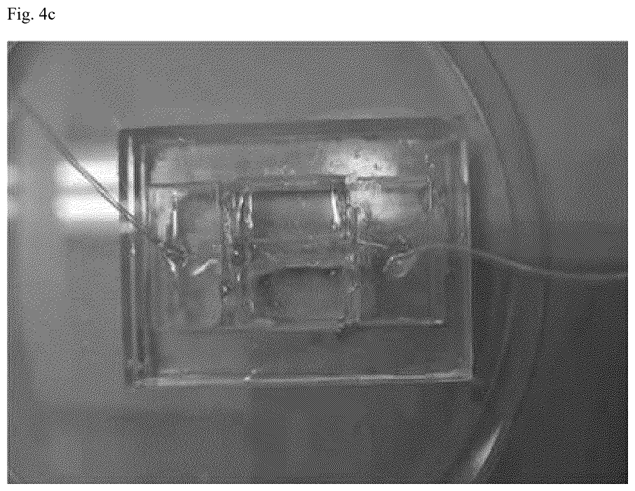

Reference is now made to FIG. 5, which is a cross-sectional view showing an Organ-Mimicking Organ-on-a-Chip according to a third embodiment including a platform having a cell culture layer, a membrane part, and an air chamber providing deformation force. According to this embodiment, the third embodiment is suitable for mimicking movement in which the cell culture layer is deformed in the vertical direction.

This embodiment includes a third cell culture layer 340, a third air chamber 310 with an open top, a third membrane part 330 blocking the top of the third air chamber 310, and a media chamber part 320 provided on the membrane part. The third cell culture layer 340 includes at least one cell layer, which mimics the structure of actual tissue.

The third cell culture layer 340 is provided on the third membrane part 330. The third air chamber 310 is located at the lower part and transmits vertical force to the upper third membrane part 330 through the injection and discharge of air to induce vertical movement of the third cell culture layer 340. The fifth air chamber 310 has a fifth space 3101 inside.

The third membrane part 330 is in the form of a PDMS membrane, blocks the top of the fifth air chamber 310, directly receives pressure change from the fifth air chamber 310, is deformed vertically, and transmits this deformation to the upper third cell culture layer 340. The media chamber part 320 is a layer for material exchange with the third cell culture layer 340 and may contain liquid medium containing nutrients and growth factors.

The media chamber part 320 provides a sixth space 3202 in which the third cell culture layer 340 can move vertically. Through this structure, the pressure change of the fifth air chamber 310 is transmitted to the third cell culture layer 340 in the media chamber through the third membrane part 330, so that mechanical stimulation in the vertical direction occurring in actual tissue can be effectively mimicked.

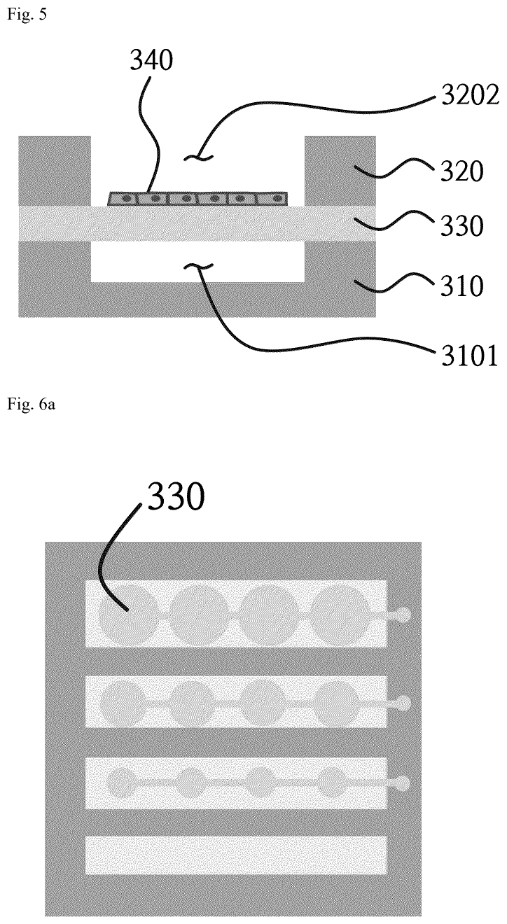

Reference is now made to FIG. 6A, which is a schematic diagram of an Organ-Mimicking Organ-on-a-Chip, and FIG. 6B is a photograph thereof. These figures show a structure that can experiment with a plurality of cell culture layers with the same membrane part size or a plurality of third cell culture layers 340 with different third membrane part 330 sizes according to the size of the air chamber.

A fourth embodiment includes a plurality of the third embodiment, but a person skilled in the art will be able to have various embodiments by variously combining the first through third embodiments.

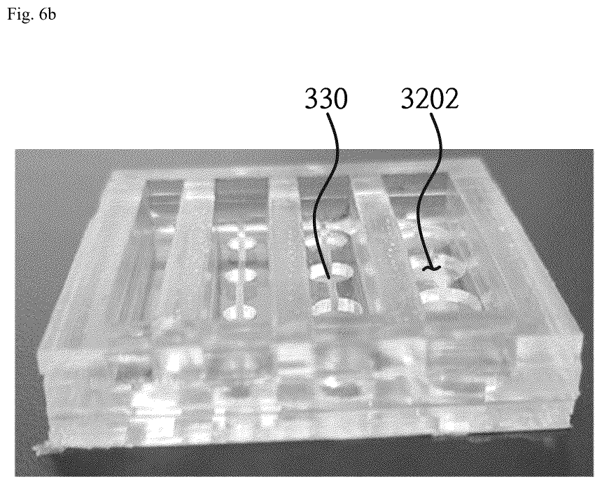

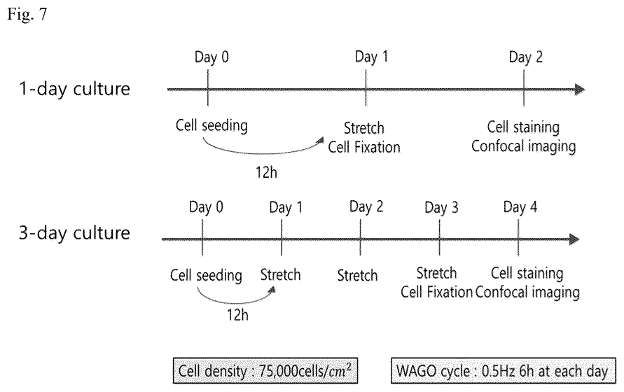

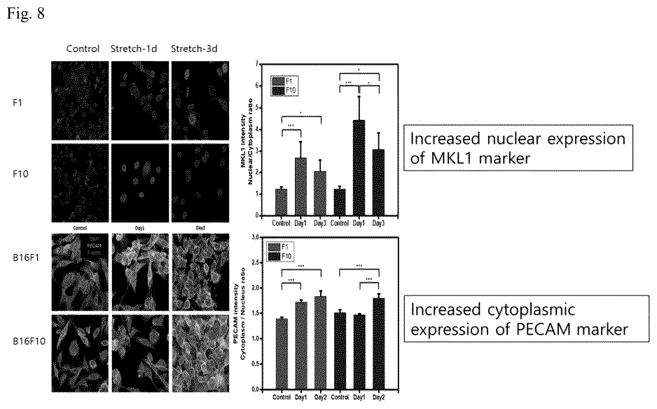

FIG. 7 shows culture conditions of a cell culture layer. FIG. 8 is a visual representation of types of mechanical stimulation that can be applied to a cell culture layer, showing patterns and changes of Stretch and Compression stimulation. FIG. 9 is a graph evaluating cell death change due to mechanical stimulation applied to cells.

In FIG. 7, Stretch stimulation is an important variable for checking how cells respond to physical stimulation, and cell changes are observed through the stretching cycle (0.5 Hz) and time (6 hours). In 1-day culture, cells are fixed and analyzed immediately after receiving stimulation, whereas in 3-day culture, it is an experimental method of observing cell reactions for a long time by giving stretching stimulation every day.

FIG. 8 shows how mechanical stimulation affects the expression of MKL1 and PECAM markers in cells, and it can be confirmed that nuclear expression and cytoplasmic expression increase as stimulation time passes. MKL1 shows a characteristic of increased expression in the nucleus, and PECAM shows a characteristic of increased expression in the cytoplasm. Therefore, it is visually confirmed that marker expression increases when cells receive mechanical stimulation.

FIG. 9 analyzes the effect of mechanical stimulation on cell death, suggesting that the response to stimulation differs depending on the characteristics of cells. Therefore, in order to reflect the mimicking of human tissue in which movements such as stretching and compression occur, two-dimensional or three-dimensional mechanical stimulation is necessary for the mimicking system.

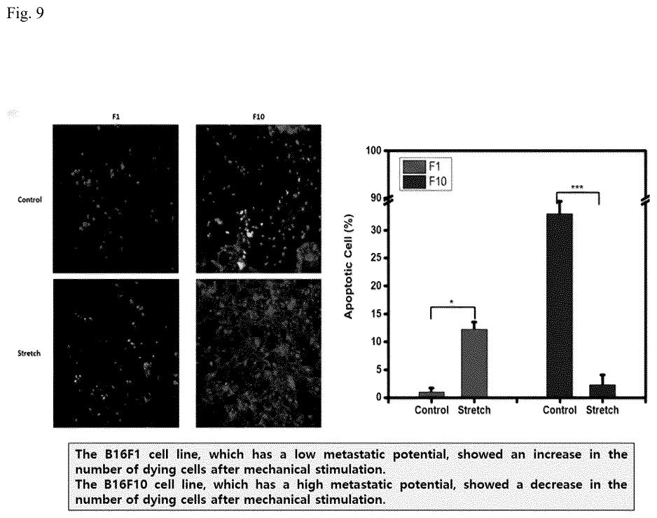

FIG. 10 is a view showing that various contractions and expansions of the cell culture layer can be confirmed as a result of experiments on a plurality of cell culture layers with different membrane part sizes according to the size of the air chamber.

### Second Aspect—Manufacturing Method

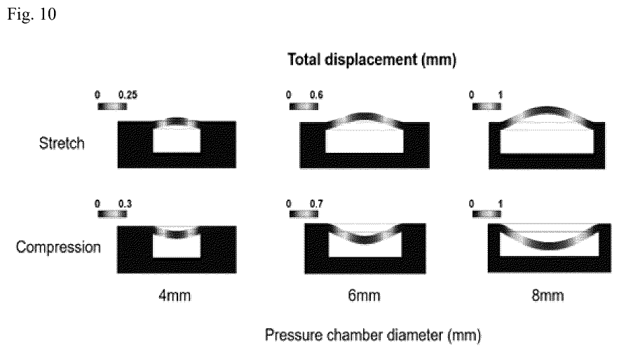

Reference is now made to FIG. 11, which is a flowchart showing a manufacturing process of an organ-mimicking organ-on-a-chip according to a second aspect of the present invention. According to this flowchart, the method for manufacturing an Organ-Mimicking Organ-on-a-Chip includes an air chamber manufacturing step, a membrane manufacturing step, a bonding step, a tubing step, and a cell culture layer formation step, and may optionally include a cutting step.

The air chamber manufacturing step is a step of forming the basic structure of the air chamber by pouring a mixture of polydimethylsiloxane (PDMS) and PDMS curing agent into a mold designed according to the target structure, and obtaining it by separating the cured PDMS structure from the mold.

It is preferable that the polymer material of the air chamber has a ratio of 8:1 to 12:1 of polydimethylsiloxane (PDMS) and PDMS curing agent. If the range is exceeded, there is a problem that the structure becomes too rigid and flexibility decreases, and if it is less than the range, there is a problem that the strength of the structure weakens and it is difficult to maintain the shape.

The membrane manufacturing step is a step of separately manufacturing a thin membrane to be used as the membrane part. At this time, a polymer is used to control the flexibility of the membrane. It is preferable that the polymer material of the membrane has a ratio of 25:1 to 35:1 of polydimethylsiloxane (PDMS) and PDMS curing agent. If the range is exceeded, there is a problem that the membrane becomes too flexible and cell support power decreases, and if it is less than the range, there is a problem that the flexibility of the membrane is insufficient and it is difficult to obtain the desired deformation.

The membrane is made by making a mold with acrylic material, spin coating PDMS to form a thin film, making a desired pattern with stamping technology. Biological materials such as dECM may be included in the pattern to promote cell attachment and growth. It goes through a membrane separation process. Through this structure and manufacturing process, the membrane mimics the structure of actual biological tissue, allowing cells to grow and function in an environment similar to the living body. This can provide a more accurate in vitro organ model and be utilized for various biological research and drug testing.

The bonding step is a step of bonding the air chamber structure and the membrane through plasma treatment. This step can be repeated several times depending on the situation, and the upper structure, lower structure, membrane part, etc. can be sequentially bonded.

The tubing step is a step of inserting tubing for air injection and discharge or media circulation at appropriate positions of the air chamber.

The cutting step, if necessary, is a step of precisely cutting out the part to be formed as an air chamber or media chamber from the bonded structure. This step can be optionally performed depending on the embodiment.

The cell culture layer formation step is a step of forming a cell culture layer on the membrane part. The process of attaching the cell layer to the membrane part includes a surface activation step and an ECM coating step.

In the surface activation step, the surface of the membrane made of PDMS or other materials is activated by treating it with oxygen plasma. This process changes the chemical properties of the membrane surface to increase hydrophilicity and increase surface energy, facilitating the attachment of ECM proteins and cell adhesion in subsequent steps.

In the ECM coating step, ECM proteins (e.g., fibronectin, collagen, laminin) are coated on the activated surface to promote cell attachment. The selected ECM protein solution is evenly applied to the membrane surface and cultured for a certain period of time to provide a microenvironment that supports cell attachment, growth, and differentiation.

For example, according to the following formula 1, the PMMA or PDMS surface is first oxidized by plasma or UV-ozone treatment to form hydroxyl groups:

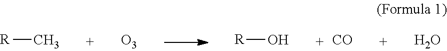

Next, the surface is silanized using APTS, and in this process, amino groups are introduced to the surface. After reacting glutaraldehyde with the silanized surface to introduce aldehyde groups, the desired protein is covalently immobilized on the surface by reacting the aldehyde groups with amino groups of the desired protein. Finally, when cells are cultured on the surface treated in this way, the cells recognize the immobilized protein layer, attach to it, and grow. Through this series of processes, the surface of synthetic polymers that originally have low cell affinity can be changed to an environment where cells can attach and grow well.

This manufacturing method is designed to be applicable to various embodiments, and specific steps can be added, modified, or omitted according to the characteristics of each embodiment. The order of steps can be adjusted considering the characteristics of the embodiment and the efficiency of the manufacturing process.

### Third Aspect-Dynamic Organ Mimicking System

A Dynamic Organ Mimicking System according to a third aspect includes: a cell culture layer; a platform including a membrane part on which the cell culture layer is seated and an air chamber providing deformation force to the membrane part; and a pressure supply unit that controls the pressure of gas provided to the air chamber.

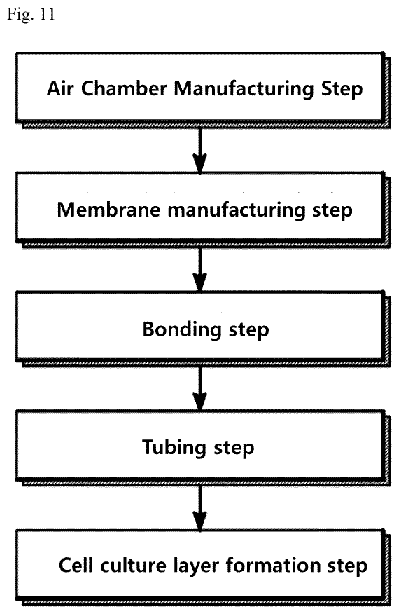

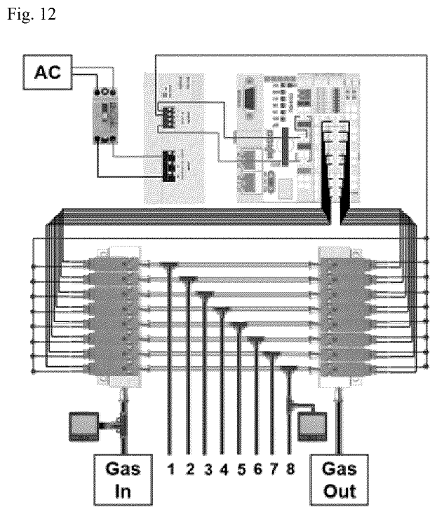

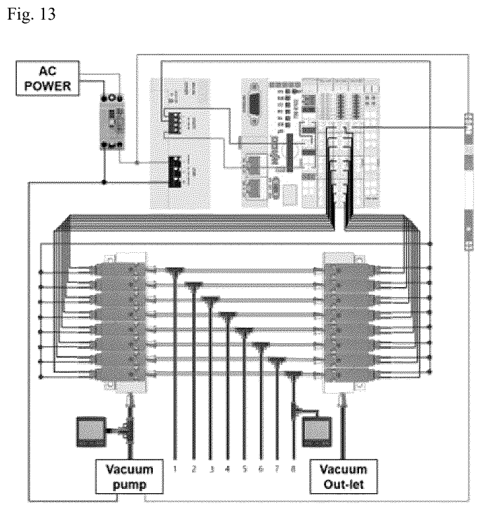

Reference is now made to FIGS. 12 and 13, which are schematic diagrams of a pressure supply unit capable of periodically applying gas and vacuum. Since the description of the cell culture membrane and the function of the platform in the Organ-Mimicking Organ-on-a-Chip has been provided above, the pressure supply unit will be described here.

The pressure supply unit includes a power supply unit, a controller, a valve, a pump, an inlet port, an outlet port, and a pressure sensor.

The power supply unit supplies power to the system, and the controller is a central control device that controls the entire system by executing programmable logic.

The valve is configured to precisely control the flow of gas flowing into or discharged from the air chamber of the Dynamic Organ Mimicking System. The valve according to the present invention precisely controls the deformation force applied to the air chamber by performing opening and closing of air flow and flow rate control.

When the Dynamic Organ Mimicking System according to the present invention mimics the lung, the valve may be a solenoid valve or a proportional control valve that operates according to a control signal input from the controller. The solenoid valve is opened and closed by an electrical signal, and can precisely mimic the inhalation and exhalation cycle of the lung mimicking system through fast response speed. In the case of a proportional control valve, since the valve opening degree is continuously controlled according to the size of the input electrical signal, more detailed flow rate control is possible.

The valve may be composed of a plurality of valves for periodic pressurization and depressurization, which independently control the flow of air or vacuum to form a pressure difference in the air chamber. It also has a backflow prevention function to prevent system errors due to gas backflow.

The operation of the valve is managed by the control algorithm of the controller, and can be set to automatically open and close according to the inhalation and exhalation cycle. Through this, pressure changes similar to the actual breathing pattern of the lung mimicking system can be reproduced.

The inlet port and outlet port are ports for supplying and discharging gas, and the pressure sensor monitors the pressure of the gas.

The pressure sensor performs the function of detecting the pressure inside the air chamber in real time and converting it into an electrical signal to transmit it to the controller.

When the Dynamic Organ Mimicking System according to the present invention mimics the lung, it is preferable that the pressure detection range of the pressure sensor measures the pressure inside the air chamber within a range of 100 to 1,500 mbar, and it is more preferable that the pressure inside the air chamber is 100 to 1,000 mbar.

The pressure sensor transmits the measured pressure data to the controller, and this data is utilized for real-time feedback control. The controller adjusts the operation of the valve and pump based on the data received from the pressure sensor to maintain the target pressure range. Through this closed-loop control method, the accurate pressure simulation function of the lung mimicking system can be performed.

The controller controls the action of gas in, gas out, vacuum in, and vacuum out by adjusting the valve and pump.

It is preferable that the pressure sensor according to the present invention includes a multi-channel synchronization function so that while a plurality of pressure sensors are operating simultaneously for complex breathing patterns or multi-organ mimicking, pressure data between each channel can be accurately compared or analyzed.

The pressure sensor according to the present invention may further include a Non-Linear Pressure Detection function. Through this, not only simple linear pressure changes but also complex periodic and aperiodic pressure patterns can be precisely detected in the Dynamic Organ Mimicking System.

The pressure supply unit can apply a closed-loop control method based on PID control (proportional-integral-derivative control) to precisely control stretching and compression operations. PID control is a method in which the controller adjusts the valve and pump in real time based on data measured by the pressure sensor to maintain the target pressure.

Reference is now made to a figure which illustrates examples of types of mechanical stimulation that can be applied by generating various waveforms by a controller. The first waveform shows that stretching and compression are alternately applied at regular intervals periodically. The second waveform shows a stimulation pattern in which stretching and compression change gradually as the frequency decreases over time. The third type shows a complex stimulation pattern in which stretching and compression occur simultaneously.

When the Dynamic Organ Mimicking System according to one embodiment mimics the lung, by organically combining the breathing pattern controlled by the pressure supply unit and the microfluidic system, gas exchange system, and electrical/mechanical stimulation system, the environment of the actual lung can be mimicked more precisely.

For example, when mimicking the breathing pattern of an asthma patient, the pressure supply unit can be set to 2 seconds inhalation plus 4 seconds exhalation (total 6 seconds cycle) reflecting the exhalation delay characteristic due to airway constriction. In the case of a sleep apnea model, the pressure supply unit can apply a cycle (total 15 seconds) that repeats rapid compensatory inhalation (3 seconds) and short exhalation (2 seconds) after breathing stops for 10 seconds.

Through this pressure supply unit, the present invention can accurately mimic the mechanical environment of various organs, and especially can effectively reproduce the dynamic characteristics of organs such as lungs where periodic expansion and contraction are important.

### Fourth Aspect—Drug Administration Device

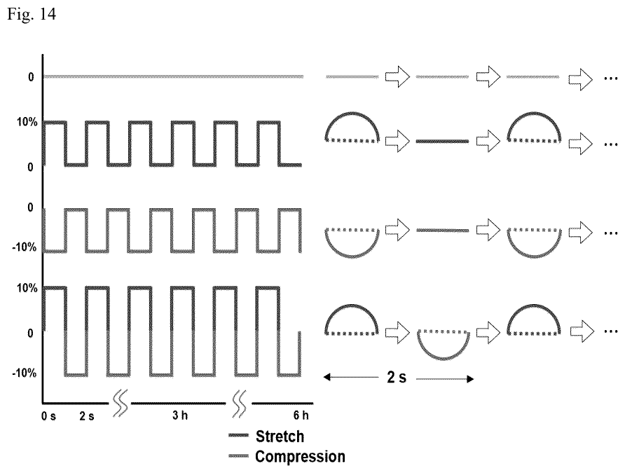

Reference is now made to FIG. 15, which is a schematic diagram of a drug administration device. According to this figure, the drug administration device includes a drug-carrying layer 410 having a drug delivery vehicle 411 inside, and a circuit configuration layer 420 having a receiving antenna 421 and a heating electrode 423 inside.

The drug-carrying layer 410 is a layer that stores drugs to be administered to the cell layer of the Dynamic Organ Mimicking System, and is a layer that directly contacts the organ mimicking system. It may be made of a first layer porous biocompatible material. This layer carries drug delivery vehicles 411 in the form of particles containing drugs, and is attached to the target site. The drug delivery vehicle 411 releases the drug in response to physical (electrical) stimulation.

The drug delivery vehicle 411 is composed of nano-sized particles. The drug delivery vehicle 411 contains a drug and is designed to release the drug by specific stimulation. The drug delivery vehicle 411 is in the form of nanoparticles designed so that the drug inside can be effectively released through polymer expansion by heating by electrical stimulation.

The drug delivery vehicle 411 may use polymeric nanoparticles and/or inorganic nanoparticles. Polymeric nanoparticles are function-controlled particles capable of tissue-customized drug delivery. Inorganic nanoparticles are mainly made of biocompatible materials including SiO2, and are drug preservation-type biodegradable particles that can protect drugs in the body for a long time and maintain activity.

The drug-carrying layer 410 is developed as a bio-friendly, tissue-friendly material with integrated physical properties with lung tissue. It is preferably made of a polymer adhesive material with high adhesion to the curved surface and microstructure of tissue.

The drug-carrying layer 410 is also designed with a porous structure, which greatly improves the efficiency and controllability of drug delivery. The holes of the porous structure have the characteristic of changing their size by responding sensitively to temperature changes. When the temperature rises due to heating of the heating electrode 423, the hole size expands and nanoparticles that were carried inside are released through the holes.

The circuit configuration layer 420 includes electronic devices that detect external stimulation and control the internal system, and through this, the drug release process is precisely controlled. This layer includes an antenna 421 for detecting electrical signals given from outside and a heating electrode 423 that can convert them into heat or electrical stimulation to induce drug release.

The antenna 421 plays a role of receiving external stimulation, for example, radio waves or electrical signals, and delivering it to the control system inside the device. The heating electrode 423 generates heat in response to the external signal received by the antenna 421. This heat is transmitted to the drug-carrying layer 410 to induce drug release.

The receiving antenna 421 and heating electrode 423 can be made of printable biodegradable conductive rubber-based ink material.

### Embodiment: Organ-on-a-Chip Structure for Lung Fibrosis Mimicking

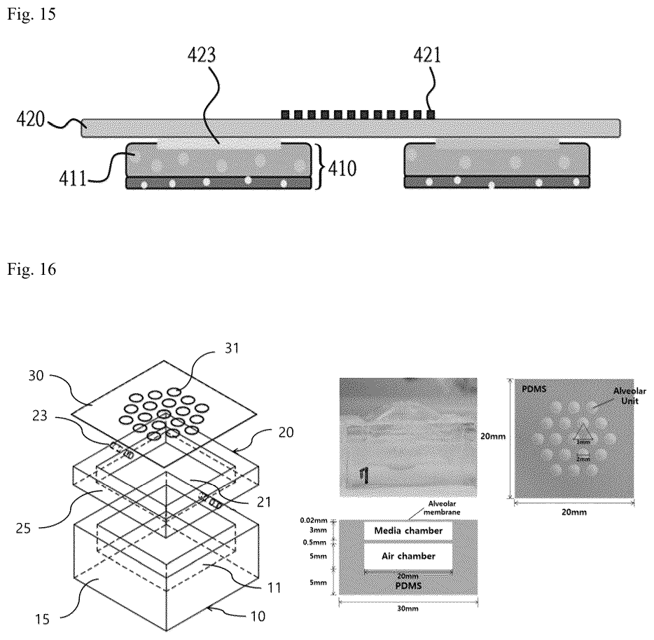

Referring to FIG. 16, an organ-on-a-chip for mimicking lung fibrosis according to an embodiment of the present invention includes an air chamber 10, a media chamber 20, an alveolar pattern membrane 30, and a cell culture layer.

The air chamber 10 has a pressure control space 11 and an air chamber frame 15 therein, and is a component that provides mechanical stimulation to mimic the respiratory movement of alveoli. The air chamber 10 is located at the lowermost end of the organ-on-a-chip and serves as a mechanical driving unit of the entire system.

It is preferable that the material of the air chamber 10 is composed of a polymer material mixed with an elastomer (for example, PDMS) and a curing agent in a ratio of 8:1 to 12:1. If the range is exceeded, the structure becomes excessively rigid and a problem occurs in which pressure transmission efficiency is lowered, and if it is less than the range, a problem occurs in which structural strength is insufficient and durability against repetitive pressure changes is lowered.

The air chamber 10 is directly bonded to the media chamber 20 and performs a function of transmitting pressure change to the upper part. In addition, it can reproduce respiratory movement occurring in actual alveoli by providing periodic mechanical stimulation to the alveolar pattern membrane 30 and the cell culture layer.

The media chamber 20 is stacked on the upper part of the air chamber 10, and is a middle layer structure in which a culture solution supply passage 23 is formed inside. The media chamber includes an internal space 21 and a media chamber frame 25, and is responsible for a life maintenance function of supplying nutrients necessary for the cell culture layer and removing metabolites.

It is preferable that the thickness of the media chamber frame 25 is set to 0.5 mm, which is to secure sufficient culture solution storage space while maintaining the compactness of the entire structure. The culture solution supply passage is designed for efficient material exchange with the cell culture layer. The culture solution is circulated and supplied through a peristaltic pump, and is replaced with new culture solution at one-day intervals to maintain a stable culture environment for cells.

The media chamber 20 performs a relay role of transmitting mechanical stimulation transmitted from the lower air chamber 10 to the upper alveolar pattern membrane 30. In addition, it can provide an environment necessary for cell survival and growth by directly contacting the cell culture layer.

The alveolar pattern membrane 30 is stacked on the upper part of the media chamber 20, and is a structure in which a plurality of pores 31 are arranged and formed on a plane. The alveolar pattern membrane 30 is a core component that implements an environment of individual alveolar units 32 by mimicking the porous structure of actual alveolar tissue.

The diameter of the pore 31 is set to 2 mm to 4 mm, and it is preferable that the pore spacing is arranged at 2 mm. These dimensions are set to ensure the independence of individual alveolar units 32 while providing sufficient cell culture space. The thickness of the alveolar pattern membrane 30 is formed to 0.02 mm, enabling effective transmission of mechanical stimulation.

The material of the alveolar pattern membrane 30 may be composed of at least one of PDMS, polyurethane, silicone, and polyimide, and the pores 31 are formed through a punching process.

These materials can maintain stable performance even under repetitive deformation by providing biocompatibility and appropriate flexibility.

The alveolar pattern membrane 30 performs a function of directly transmitting pressure change transmitted from the lower air chamber 10 and media chamber 20 to the cell culture layer. Each pore 31 acts as an independent alveolar unit 32, enabling measurement of individual fibrosis progression state.

The cell culture layer is a biological component that is filled inside each pore 31 provided in the alveolar pattern membrane 30 and includes lung cells. The cell culture layer is composed of a hydrogel matrix and is responsible for a function capable of observing fibrosis progression state by reproducing an actual alveolar environment.

The hydrogel matrix may be composed of at least one of GelMA, collagen, alginate, chitosan, PEG, and PEGDA. In particular, it is preferable that the mixing ratio of GelMA and Gelatin is set to 8:4 to 8:8, and more preferably, it is set to 8:5 to 8:7.

The lung cells are composed of a multi-cell layer including lung epithelial cells, lung endothelial cells, and fibroblasts. This cell composition can observe cell-to-cell interactions appearing in the fibrosis process by reproducing the cellular environment of actual alveoli.

The cell culture layer survives by receiving nutrients supplied from the media chamber 20 in the pores 31 of the alveolar pattern membrane 30, and exhibits physiological changes related to fibrosis in response to mechanical stimulation transmitted from the air chamber 10. The cell culture layer located in each pore 31 acts as an independent experimental unit, enabling individual analysis.

### Hydrogel Matrix Composition

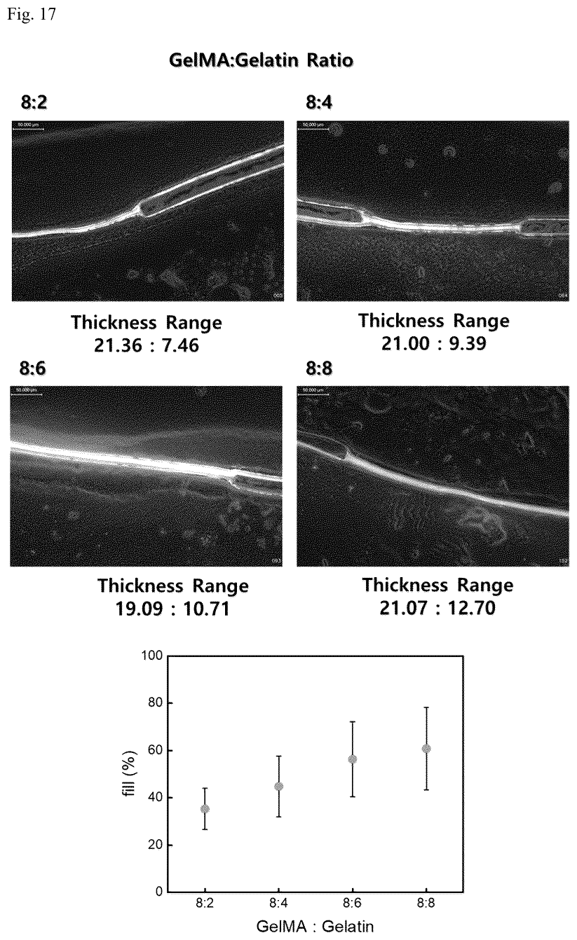

Referring to FIG. 17, membrane formation characteristics according to the composition ratio of the hydrogel matrix constituting the cell culture layer were evaluated. Fill ratio and membrane thickness were measured while changing the mixing ratio of GelMA and Gelatin to 8:2, 8:4, 8:6, and 8:8.

As a result of the experiment, when the GelMA: Gelatin ratio was 8:2, the fill ratio was about 35%, and the membrane thickness was measured as 21.36±7.46. At an 8:4 ratio, the fill ratio increased to about 45%, and the membrane thickness was confirmed as 21.00±9.39. At an 8:6 ratio, the fill ratio was about 55-60%, and the membrane thickness was measured as 19.09±10.71. At an 8:8 ratio, the fill ratio maintained about 60%, but the membrane thickness was measured as 21.07±12.70.

When the Gelatin ratio is low below the 8:4 range, the structural stability of the hydrogel is insufficient, making it difficult to maintain the shape of the matrix during long-term culture, and a problem occurs in which the fill ratio becomes significantly low. When exceeding 8:8, the mechanical strength of the hydrogel increases excessively, inhibiting normal cell growth and differentiation, and the membrane thickness uniformity improvement effect is saturated, making it difficult to expect additional performance improvement.

Therefore, it is preferable to set the mixing ratio of GelMA: Gelatin to 8:6 to 8:8, which is a condition that simultaneously satisfies stable cell culture layer formation in the pores 31 and provision of an appropriate cell growth environment.

### Method for Measuring Mechanical Properties of Alveolar Membrane

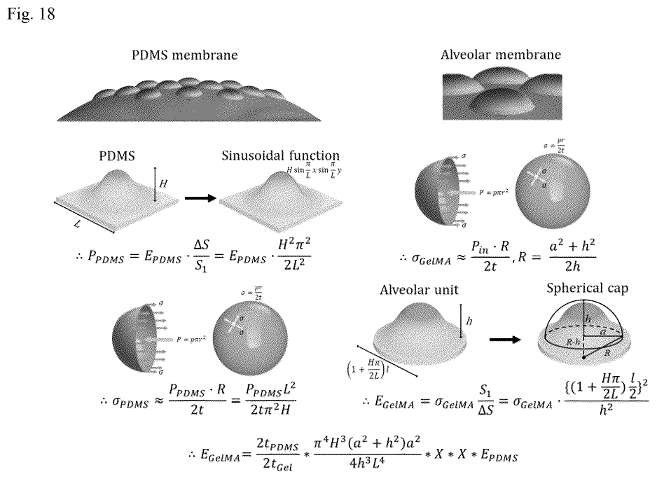

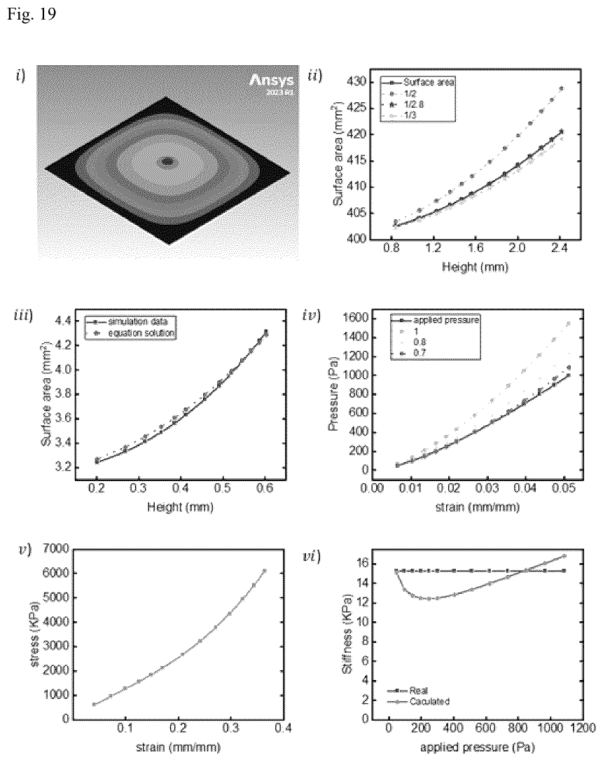

Referring to FIGS. 18 and 19, the method for measuring mechanical properties of the alveolar membrane according to the present invention includes an observation step, a modeling step, and a calculation step.

The observation step is a step of observing deformation of the alveolar pattern membrane due to pressure change of the air chamber by imaging. Pressure in a range of 0.1 kPa to 5 kPa is periodically applied to the air chamber through a pressure control unit. The pressure cycle is set to 0.2 Hz to 1 Hz to mimic the actual respiratory cycle.

When the pressure is less than 0.1 kPa, a problem occurs in which the deformation of the alveolar pattern membrane is insignificant and measurement accuracy is lowered. When the pressure exceeds 5 kPa, a problem occurs in which damage occurs to the cell culture layer due to excessive deformation and deviates from actual physiological conditions.

Imaging is performed in real time using a high-resolution microscope, and the deformation pattern of each alveolar unit is independently recorded. Image capture is performed continuously at 1-second intervals to accurately capture the temporal change of deformation. Geometric parameters such as deformation height (H, h), deformation radius (a), and alveolar unit spacing (L) are extracted from the captured images.

The modeling step is a step of analyzing the deformation of the alveolar pattern membrane and the cell culture layer into mathematical models respectively. This step includes elastomer membrane modeling and alveolar membrane modeling.

In elastomer membrane modeling, the deformation of the alveolar pattern membrane is modeled as a sinusoidal function. The deformation function is expressed as H sin(πx/L)×sin(πy/L), where H represents the deformation height and L represents the alveolar unit spacing. When using PDMS, which is an embodiment of an elastomer, the stress-strain relationship is calculated as σ_elastomer=E_elastomer×(H²π²)/(2L²).

In alveolar membrane modeling, the deformation of the cell culture layer is modeled as a spherical cap model. The curvature radius R of the deformed alveolar membrane is calculated as (a²+h²)/(2h), where a represents the deformation radius and h represents the deformation height. Through this, the stress of the alveolar membrane is calculated as σ_GelMA=P_in×R/(2t).

The two modeling results are interrelated, and mathematically express the relationship in which the deformation of the elastomer membrane directly affects the deformation of the cell culture layer.

The calculation step is a step of quantitatively calculating the Young's modulus of the cell culture layer based on the modeling results. In this step, the mechanical properties of the hydrogel matrix are calculated as shown in Formula 2 below using the following equation.

$$E\_hydrogel = ( 2 t\_polymer membrane / 2 t\_cell layer ) \times ( {π}^{4} {H}^{3} ( {a}^{2} + {h}^{2} ) {a}^{2} ) / ( 4 {h}^{3} {L}^{4} ) \times E\_polymer member \qquad ( Formula 2 )$$

Where each parameter is as follows:
E_hydrogel: Young's modulus of the cell culture layer (kPa)
t_polymer membrane: Thickness of the alveolar pattern membrane (0.02 mm)
t_cell layer: Thickness of the cell culture layer (measured in the observation step)
H: Elastomer deformation height (measured in the observation step)
L: Alveolar unit spacing (2 mm)
a: Deformation radius (measured in the observation step)
h: Deformation height (measured in the observation step)
E_polymer membrane: Young's modulus of the alveolar pattern membrane (known value)

When using PDMS as an embodiment of the elastomer, the Young's modulus value of PDMS is applied for E_polymer membrane.

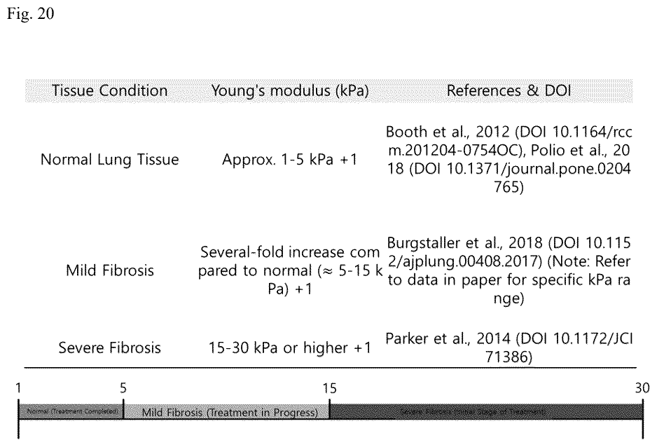

The calculated Young's modulus value is used to determine the fibrosis progression state according to the criteria presented in FIG. 20. Normal lung tissue exhibits a value of 1-5 kPa, mild fibrosis exhibits 5-15 kPa, and severe fibrosis exhibits a value of 15-30 kPa or more.

Through this series of measurement processes, the mechanical properties of each alveolar unit can be quantified in real time, and individual fibrosis progression state analysis is possible.

The features, structures, effects, etc. illustrated in each of the above-described embodiments can be combined or modified for other embodiments by a person having ordinary knowledge in the field to which the embodiments belong. Therefore, contents related to these combinations and modifications should be interpreted as being included in the scope of the present invention.

The reference numerals used herein are as follows:
110: First air chamber; 1101: First tube; 1102: First space
120: Second air chamber; 1201: Second tube; 1202: Second space
130: First membrane part; 140: First cell culture layer
150: Microfluidic supply unit; 160: Gas exchange unit
170: Electrical/mechanical stimulation unit
210: Third air chamber; 220: Fourth air chamber
230: Second membrane part; 240: Second cell culture layer
310: Fifth air chamber; 320: Media chamber part
330: Third membrane part; 340: Third cell culture layer
410: Drug-carrying layer; 411: Drug delivery vehicle
420: Circuit configuration layer; 421: Receiving antenna
423: Heating electrode
10: Air chamber
15: Air chamber frame
20: Media chamber
25: Media chamber frame
30: Alveolar pattern membrane
31: Opening

What is claimed is:

1. An Organ-Mimicking Organ-on-a-Chip comprising: a cell culture layer; and a platform including a membrane part on which the cell culture layer is seated and an air chamber providing deformation force to the membrane part.

2. The Organ-Mimicking Organ-on-a-Chip of claim 1, wherein the cell culture layer is capable of two-dimensional or three-dimensional deformation according to the deformation of the membrane part.

3. The Organ-Mimicking Organ-on-a-Chip of claim 1, wherein the platform is manufactured by mixing polydimethylsiloxane (PDMS) and a curing agent.

4. The Organ-Mimicking Organ-on-a-Chip of claim 1, wherein the membrane part is more flexible than the air chamber.

5. The Organ-Mimicking Organ-on-a-Chip of claim 1, wherein the membrane part is manufactured by mixing polydimethylsiloxane (PDMS) and a curing agent in a composition ratio of 25:1 to 35:1.

6. The Organ-Mimicking Organ-on-a-Chip of claim 1, wherein the air chamber is manufactured by mixing polydimethylsiloxane (PDMS) and PDMS curing agent in a composition ratio of 8:1 to 12:1.

7. The Organ-Mimicking Organ-on-a-Chip of claim 1, wherein the air chamber includes a first air chamber and a second air chamber, and the first air chamber and the second air chamber are located on both sides of the membrane part to provide deformation force in a lateral direction to the cell culture layer.

8. The Organ-Mimicking Organ-on-a-Chip of claim 1, further comprising a third space and a fourth space on both sides of the membrane part, wherein the third space and the fourth space induce more uniform deformation in the cell culture layer.

9. The Organ-Mimicking Organ-on-a-Chip of claim 1, wherein the air chamber is located at a lower end of the cell culture layer, and a media chamber containing liquid medium in which material exchange between the membrane part and cell layer occurs is provided on the air chamber so that the cell culture layer can be deformed in a vertical direction.

10. The Organ-Mimicking Organ-on-a-Chip of claim 9, wherein the Organ-Mimicking Organ-on-a-Chip includes a plurality of the air chambers, the membrane parts, and the media chambers.

11. The Organ-Mimicking Organ-on-a-Chip of claim 10, wherein sizes of the air chambers are different from each other, and the plurality of membrane parts vary according to the size of the air chamber.

12. The Organ-Mimicking Organ-on-a-Chip of claim 10, wherein each of the air chambers is independently controlled to simultaneously provide different physical stimulation to the plurality of cell culture layers.

13. The Organ-Mimicking Organ-on-a-Chip of claim 1, further comprising a microfluidic supply unit that supplies culture solution containing nutrients and oxygen necessary for the cell culture layer.

14. The Organ-Mimicking Organ-on-a-Chip of claim 13, further comprising a gas exchange unit for controlling oxygen (O₂) and carbon dioxide (CO₂) concentration of the cell culture layer.

15. The Organ-Mimicking Organ-on-a-Chip of claim 14, further comprising an electrical/mechanical stimulation unit that provides electrical or mechanical stimulation for growth and functional improvement of the cell culture layer.

16. The Organ-Mimicking Organ-on-a-Chip of claim 15, wherein the air chamber, a microfluidic system, a gas exchange system, and the electrical/mechanical stimulation unit are configured to mimic disease-specific breathing patterns, and the air chamber reproduces physiological conditions of a specific disease by controlling respiratory cycle and pressure change.
